(19) United States
(12) Patent Application Publication
(10) Pub. No.: US 2015/0190538 A1
(43) Pub. Date: Jul. 9, 2015

(54) Sanitizing Systems and Methods

(71) Applicants: Robert E. Olvera, San Antonio, TX (US); Jerry A. Lochridge, Conway, AR (US); Mark L. Berrier, Austin, TX (US)

(72) Inventors: Robert E. Olvera, San Antonio, TX (US); Jerry A. Lochridge, Conway, AR (US); Mark L. Berrier, Austin, TX (US)

(21) Appl. No.: 14/592,458

(22) Filed: Jan. 8, 2015

Related U.S. Application Data
(60) Provisional application No. 61/925,166, filed on Jan. 8, 2014.
(60) Provisional application No. 61/939,765, filed on Feb. 14, 2014.

(51) Int. Cl. A61L 2/10; A61L 2/24; A61L 2/18

(52) U.S. Cl. CPC A61L 2/10; A61L 2/18; A61L 2/24

(57) ABSTRACT

Systems and methods for disinfecting/sanitizing various articles using disinfecting solutions and ultraviolet (UV) light. In one embodiment, an exemplary system includes a tank that contains a sanitizing liquid, coupled to a pipe system that includes multiple spray nozzles. A pump pumps the sanitizing liquid from the tank to the nozzles, which dispense the liquid to a disinfecting area. Some or all of the system may be mounted on a transportable mobile unit such as a trailer. In one embodiment, a portion including the tank and pump are mounted on the mobile unit and are connectable to a pipe system and nozzles that are permanently installed in a disinfecting area, such as the interior of a vehicle. Other embodiments are also possible.

### CROSS-REFERENCE TO RELATED APPLICATIONS

This application claims the benefit of U.S. Provisional Patent Application 61/925,166, filed Jan. 8, 2014 by Robert E. Olvera, et al. and U.S. Provisional Patent Application 61/939,765, filed Feb. 14, 2014 by Robert E. Olvera, et al. All of the foregoing patent applications are incorporated by reference as if set forth herein in their entirety.

### BACKGROUND

The invention relates generally to systems and methods for sanitizing.

Anywhere you have a tremendous amount of people interacting on a daily basis, you are subject to come in contact with any number of germs, viruses (cold and flu), plus things you can't even recognize.

No area can ever be a 100% germ free environment, but they can become much more healthy for everyone if, on a regular basis, they are subject to disinfecting and sanitizing. The public, however, may not be ready to have someone constantly wiping down tables, chairs, cabinets, walls, doorknobs, drinking fountains, etc. with disinfectants. But, as the old saying goes; “out sight, out of mind”. If locations where we have human interaction could provide environments that are routinely disinfected or sanitized without people directly knowing about it, it will become accepted, expected and even demanded.

For example, consider doorknobs or other door handles. Almost every door has one, whether it is an entrance to an office, restaurant, your home, etc. Every day, people try to avoid touching doorknobs with their bare hands in order to avoid germs. They use hand towels, elbows, handkerchiefs, sleeves of their shirts, etc. to open or close doors without touching the doorknobs. This is inconvenient and probably not even very effective, so there must be a better way to deal with germs.

### SUMMARY OF THE INVENTION

This disclosure is directed to systems and methods for disinfecting (/or sanitizing) various articles using disinfecting solutions and/or ultraviolet (UV) light.

### BRIEF DESCRIPTION OF THE DRAWINGS

Other objects and advantages of the invention may become apparent upon reading the following detailed description and upon reference to the accompanying drawings.

FIG. 1 is a diagram illustrating a trailer-mounted sanitizing system in one embodiment.

FIG. 2 is a diagram illustrating a hand-cart-mounted sanitizing system in one embodiment.

FIG. 3 is a diagram illustrating a sanitizing system installed in a home air duct system in one embodiment.

FIG. 4 is a diagram illustrating a sanitizing system installed in an aircraft air duct in one embodiment.

FIGS. 5 and 6 are diagrams illustrating a shopping cart sanitizing system in one embodiment.

FIGS. 7-9 are diagrams illustrating an escalator sanitizing system in one embodiment.

FIG. 10 is a diagram illustrating an elevator sanitizing system in one embodiment.

FIGS. 11 and 12 are diagrams illustrating a doorknob sanitizing system in one embodiment.

FIG. 13 is a diagram illustrating a toilet sanitizing system in one embodiment.

FIG. 14 is a diagram illustrating a bus sanitizing system in one embodiment.

FIG. 15 is a diagram illustrating an aircraft sanitizing system in one embodiment.

FIGS. 16-22 are diagrams illustrating a small and portable device sanitizing system in one embodiment.

FIGS. 23-31 are diagrams illustrating a countertop sanitizing system in one embodiment.

FIGS. 32-34 are diagrams illustrating a refrigerator sanitizing system in one embodiment.

While the invention is subject to various modifications and alternative forms, specific embodiments thereof are shown by way of example in the drawings and the accompanying detailed description. It should be understood, however, that the drawings and detailed description are not intended to limit the invention to the particular embodiment which is described. This disclosure is instead intended to cover all modifications, equivalents and alternatives falling within the scope of the present invention as defined by the appended claims. Further, the drawings may not be to scale, and may exaggerate one or more components in order to facilitate an understanding of the various features described herein.

### DETAILED DESCRIPTION OF EXEMPLARY EMBODIMENTS

One or more embodiments of the invention are described below. It should be noted that these and any other embodiments described below are exemplary and are intended to be illustrative of the invention rather than limiting.

As described herein, various embodiments of the invention comprise systems and methods for disinfecting/sanitizing various articles using disinfecting solutions and ultraviolet (UV) light.

With the SGBS Germinator Disinfecting Spray System, the sanitizing of doorknobs, handles and other items that are frequently touched by different people can be accomplished on a very regular basis; actually as often as every time someone uses the item. The Germinator System can have disinfecting lines ran to just about any object that one wishes to sanitize on a regular basis. The spray nozzle for the system can be built right into the door frame area and using the SGBS Logic Controller, with its modern electronics to observe when someone has used the doorknob or handle, it can signal the system to spray the area with a disinfecting solution, which will dry within a few seconds.

Other areas where the Germinator would be ideal are the bathroom water closet area, sinks, showers, countertops, laundry rooms, and in the kitchen you have sinks and countertops, as well as many other areas. Currently, in order to sanitize appliances throughout the kitchen area, it would require a portable unit to be rolled throughout the area and spray all those areas where a built-in system can't handle. (By the way, it just so happens that SGBS has developed a portable unit to be used where the built in system hasn't or can't be installed.) However, it is the intention of SGBS Sanitizing Company to work with appliance manufacturers to have the spray nozzle and delivery system built into future units. That way all that is needed is a port to the Germinator Master Distribution Unit and disinfecting and cleaning becomes very, very simple.

In new construction units, the system would be built in the walls and concealed away from view and possible damaging conditions. With existing facilities the system can be retrofitted throughout the unit and in most cases still have the majority of the system hidden from view.

Basically the SGBS Germinator System can provide a sanitizing solution for all those places people must touch with their hands or breathe the air in a crowd of people.

The SGBS Germinator System's recommended solution for these human interaction locations is the use of a two tiered approach: air duct sanitizing system and an automated overhead pipe sanitizing system.

Air ducts are generally out of sight. All that most people ever see are the air vents overhead. Usually the only time they are aware of them is when they are blowing cool or warm air.

By installing a disinfecting and sanitizing delivery system into the air duct system, it is possible to deliver a healthy environment without directly alerting people within the facility.

By providing the air duct network with a burst of disinfecting and sanitizing agent on a timed basis, while the air conditioning or heating system is running, the air circulating throughout the area will be kept disinfected and clean without anyone knowing that it is happening. If a scented air freshener is added to the spray people would assume that the owners are just trying to freshen up the room.

In cases where relying on just the air duct system would not provide a complete solution to the issue, the second tier would be incorporated.

An overhead pipe sanitizing system, which is installed much like a fire sprinkler system, would, on those occasions where the area is extremely crowded, provide additional disinfecting and sanitizing for the entire area.

The overhead pipe sanitizing system can and will be extended through the walls to as many areas as can be thought of; i.e. That hard to clean area around the water closet in the bathroom. With the Germinator System installed to several spray nozzles (built into the wall) surrounding the water closet, the Controller can signal the system to keep this area sanitized all day long.

The automated system would be programmed to provide a short spray for only a second or two every few minutes. Again, by adding a scented air freshener along with the disinfectant and sanitizing mist, people would not be alarmed.

Upgrading the air duct system would be accomplished simply, and in most occasions, without alarming anyone. Most people would think that maintenance was being performed on the system and never give it a second thought.

Installing an automated overhead piping system would require more time, and most likely would appear to be an enhancement to the fire sprinkler system.

Both the air duct and the overhead systems can be handled as a retrofit or during new construction.

The SGBS Sanitizing System consists of only a few components:
a) Primary Dispensing Unit
b) Distribution Piping System
c) Adjustable Spray Nozzles
d) Master Logic Controller
e) An accepted EPA, Federal, State, and Local Disinfecting and Sanitizing Agent (Note—there are numerous of these in use today)

Where can this system be installed and used to make the public areas a healthier and cleaner environment?

1. Commercial Transportation:
a. Airplanes and Airports
b. Buses and bus Terminals
c. Cruise Ships and Terminals
d. Trains and Stations
e. Subways and Stations
f. Taxi Cabs and Limos

2. Education Facilities:
a. School Classrooms
b. College Classrooms
c. College Dorms
d. School Buses
e. Gyms
f. Basketball Courts

3. Public Gathering Places:
a. Malls
b. Theaters
c. Sports Arenas
d. Churches
e. Bars
f. Restrooms
g. Restaurants
h. Health Centers

4. Medical Facilities
a. Doctor and Dental Offices
b. Waiting Rooms
c. Operating Rooms
d. Patient Treatment Rooms
e. Hospitals
f. Emergency Rooms

5. Hotel and Motels:
a. Reception Area
b. Hallways
c. Sitting Areas
d. Rooms
e. Exercise Rooms

6. Elevators and Escalators

7. Homes, Apartments, Condos, etc.

As indicated, areas that can benefit from and be helped with the SGBS Sanitizing System are just about endless.

Key Points of Exemplary System:

General Notes Concerning Sanitizing Systems:

Follow-up research clearly indicates that the use of the UV-C light spectrum has a lot of hazards associated with it and also, due to the types of materials that would be needed to house this lighting system, its ability to kill germs is almost totally eliminated.

All UV lighting systems clearly indicate that they can cause Skin and Eye damage is not properly controlled, and or if the power level is too high. In addition, most UV-C light will not pass through glass, ceramic, metal and or plastic. It is also highly recommended that UV lighting not be touched or viewed when it is on unless wearing the proper type of protection.

Another drawback to UV light sanitizing systems is the requirement to replace the UV bulb on a regular basis. These bulbs lose their intensity and thus their germ killing capabilities rather quickly. By the time the light has reached its half-life, it needs to be replaced. Most bulbs have less than a 2000 hour rating, which means at around 1000 hours or 40 to 42 days, these lights need to be replaced. UV lights are not cheap. To replace these bulbs will also require a lot of work in most cases.

Please note that the SGBS Sanitizing Systems are designed to utilize many different types of sanitizing liquids and in some cases, can use a powder type sanitizer. Where a powder sanitizer is called for, a different pressure controlled, spray nozzle system will be deployed.

The SGBS liquid sanitizing spray systems will utilize various types and sizes of spray nozzles from a very fine mist to a heavy spray coating. The exact nozzle will be chosen per application.

Sanitizing solution being used must be EPA, Federal, State and local approved prior to implementation. Each end user shall have the right to select the actual sanitizing solution they will use. SGBS will provide a list of authorized and approved solutions, as well as offer its own brand name solution.

Final note to existing sanitizing systems being used is that some may utilize a motion sensor, some may utilize an occupancy detector, some manual push button releases, some dry air fans, some may use timers, but at this time, research has failed to locate any sanitizing system, for the systems describe here, that utilizes all of these combined units to control and protect the public from false sprays.

Mobile Systems:

Basic DOT Version:
Self-contained, trailer mounted dispensing system, ranging in sizes from 50 gallons to over 200 gallons of sanitizing solution.
Distribution portion of the Unit is built into the element being sanitized; i.e. interior cabins of commercial buses, school buses, etc.
System uses quick connect/disconnect heads between dispensing and distribution unit for ease of hookup.
Dispensing system sits on a spill catch pan to contain any leakage from the holding tanks.
Unit can be controlled either manually or remotely through a hand-held computer controller.
System is equipped with a Fault alarm system and emergency shutdown override button.
Unit designed to run from on-board auxiliary generator or from local 120 VAC power.

Industrial-Commercial Systems:
The 50/50 gallon system is designed to mount on either a standard truck bed or on a self-contained trailer pulled behind the truck.
The 200/200 gallon system is designed to be trailer mounted only.
Trailers are designed to be connected to any standard 2 inch trailer ball.
Everything required to perform the functions of sanitizing and mildew/mold eradication is contained within these systems.
The solutions necessary to perform the functions of sanitizing and mildew/mold eradication are kept separated at all times via separate tanks.
Each system utilizes a quick connect-disconnect spray nozzle system to allow for easy change out of the spray heads for different flow requirements.
Each System has its own auxiliary power generator to allow the systems to be used even during power restoration activities such as those after a hurricane.
Each tank is set inside a safety bin to catch the solutions in the event of a major leak or damage to tank.
Each System has both manual and remote controlled activation systems with emergency shut-off capabilities.

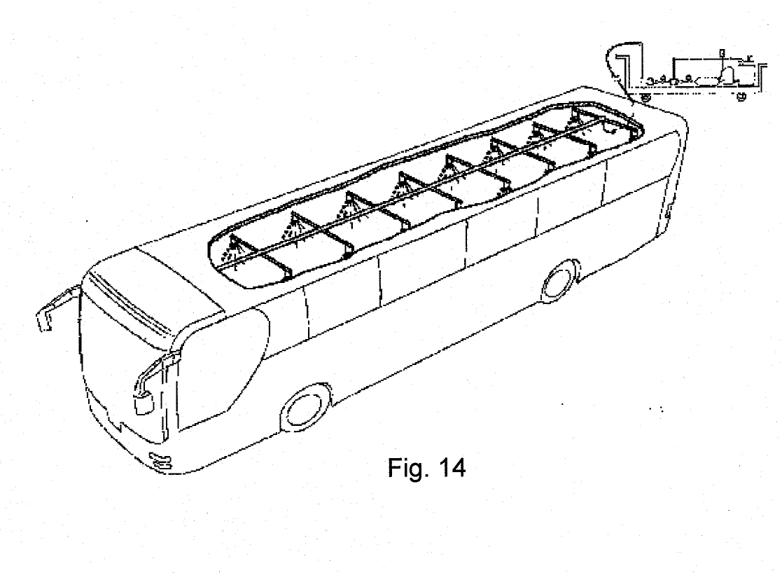

School Bus-Commercial Bus-Subway-Train Systems (e.g., FIG. 14):
This is a two part system consisting of a separate dispensing and distribution unit.
The dispensing unit is either a fixed system located close to a maintenance bay or a mobile unit constructed on a standard trailer bed.
The distribution system is pre-installed in the unit to be sanitized.
A quick connect-disconnect interface is used to tie the units together when sanitizing a unit.
Distribution system is designed to be tamper proof.
Safety systems built-in requires that an operator be in direct control of the system before sanitization can begin.
The on board distribution system will be built-into the vehicle body works so as to look like a sprinkler type installation.
Activation is a manual control using the handheld unit or remotely using a computer controlled remote carried on board the unit being sanitized.
Sanitization takes place on when no one is in the direct sanitizing area.

Taxi and Limo Systems:
Currently there are no sanitizing systems installed in taxi or limos.
The system is activated by a push button controlled by the driver.
System designed to use a fine mist nozzle in the passenger area and a slightly larger dispensing nozzle for the luggage area.
The dispensing and distribution units are designed as one and installed in the truck of the vehicle.
System design prevents accidental activation of system by covering the push button in the cab area with a lift shield cover.
The dispensing bottle is a sealed and tamper proof design . . . . The unit is screwed into the distribution system, which breaks the tamper proof seal.

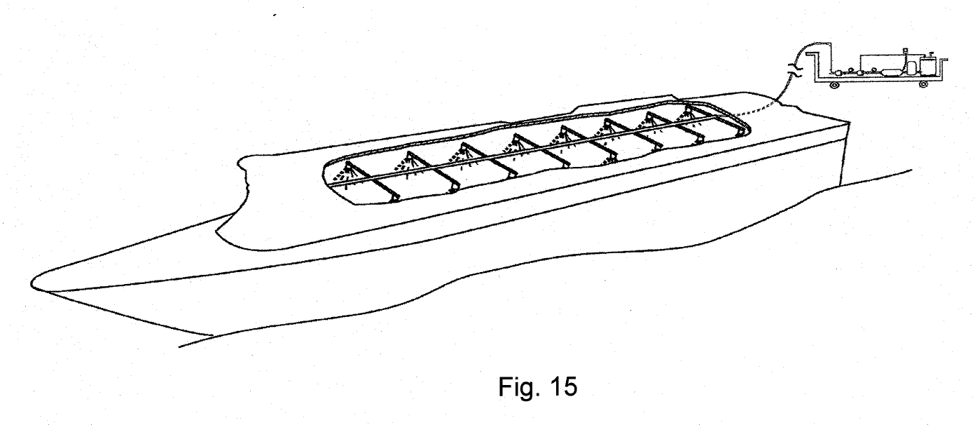

Cruise Ships (FIG. 15):
Cruise ships today use a room-to-room employee type sanitizing solution throughout the ship.
SGBS's system is a computer controlled system, installed throughout each deck of the ship.
Each deck has a sub-controller, tied back to the primary controller and can be activated as a stand-alone or as a total system sanitizing system.
SGBS uses multiple distribution units placed inside secure bulkhead areas to maintain ships water tight requirements.
All cabling and piping are run through secure bulkhead channels.
System uses Quick Connect-Disconnect spray distribution connections that allows sectionalized shut-down in the event of problems with the system.
Each dispensing unit is fitted inside an environmental containment pan to prevent large area spills of sanitizing solution.
System is equipped with many safety features that provide for quick and emergency shutdown for anywhere within the ship. In the event of an emergency with the system, a single push-button shut-off connection is installed throughout the ship that can be activated by the ship's crew.
Each sub-system of the overall system has its own monitored power connection to the ships main power system.
The entire system can be locally controlled or remotely controlled using handheld devices.

House Portable System:
Small, self-contained system, about the size of a standard vacuum cleaner, weighing less than 30 pounds and is powered by standard house current.
System designed to allow for quick changing of spray nozzle heads for different areas of the house and different jobs within those areas.
Has a retractable 15 foot hose for easy use in close areas.
Safety child proof cap used on the sanitizing solution container.
System is designed to be extremely quiet and easy to roll around the house.
Emergency shut-off switch in the event of a hose break.
Spray is activated via the hand held unit by squeezing the switch.
Sanitizing solution container is designed for easy removal for cleaning.

Home Systems:

Bathroom-Laundry Room-Kitchen-Living/Family Room:
System utilizes electronic motion detectors and occupancy detection systems and touch detection systems to activate system.
System is mounted above ceiling and only spray heads penetrate the ceiling tile, much like a sprinkler system.
Distribution system is attached to the centralized dispensing unit, usually located in the utility room or garage.
The system is developed in two separate concepts:
   Generalize spray for the entire area
   Localized spray directed toward more used areas
The generalized spray is controlled by either a timer system or manual intervention after generalized cleaning around the house.
The localized spray is controlled via motion sensors, occupancy detection sensors, touch sensors, and IR sensors, and is installed around high use areas such as the water closet, sinks, doors, etc.
System can be integrated into the SGBS Smart Home Controller System.

HVAC System:
System utilizes electronic motion detectors, occupancy detection systems and touch detection systems to activate system.
Sanitizing distribution system is physically mounted inside of Air Duct System.
Dispensing section of system is located in the utility room or garage area.
Larger, cleaning spray nozzles are used to dispense solution inside the Air Duct.
Fine mister nozzles are used at the output of the Air Duct Vents.
The spray nozzles inside the air duct system are timer controlled with a manual intervention capability.
The mister nozzles are controlled via motion and occupancy sensors as well as a timer mode.
System can be integrated into the SGBS Smart Home Controller System.
A manual override to the system allows for additional releases of the sanitizing solution.

Doorknobs-Handles-Locks (FIGS. 11-13
System utilizes electronic motion detectors, touch sensors and occupancy detection systems to activate system
Dispensing system is located in the utility room or garage area.
Distribution system is built-into the door facing or the actual door.
Touch sensors detect when someone has touched the doorknob, handle or lock and signals the controller to begin to activate system.
Occupancy detectors, IR detectors and motion sensors determine when no one is in the direct line of the spray and will allow system to sanitize the area.
System is part of the SGBS Smart Home and is controlled through the Home's Controller System.
Manual override to system allows for increased sanitizing during peak flu and other virus seasons.

Commercial Locations:

Conference Rooms, Meeting Rooms, Training Rooms, etc.:
System utilizes electronic motion detectors and occupancy detection systems to activate system in common areas such as hallways, meeting areas and lobbies.
System is mounted above ceiling and only spray heads penetrate the ceiling tile, much like a sprinkler system.
Primary activation system is a push button control located in the room that is pushed when all other cleaning of room has been accomplished. The push button is protected by a lockable cover plate with select personnel having the key.
System can also be controlled remotely via a computer based system that can be programmed to activate a single room or several rooms or a complete system spray.
System can be programmed to activate based on motion sensors and occupancy detectors so room can be sanitized after each use and over a break or lunch period.

Medical and Dental Facilities:
System utilizes electronic motion detectors and occupancy detection systems to activate system in common areas such as hallways and lobbies.
System is mounted above ceiling and only spray heads penetrate the ceiling tile, much like a sprinkler system.
Each treatment room is equipped with a manual push button activation system that sanitizes the entire room area once clean-up has been accomplished and prior to next patient entering room.
Instrument areas will have directed sanitizing spray heads that can be concentrated to a very localized area where instruments are placed.
Areas requiring continuous sanitizing, such as medicine cabinets, countertops, sinks, etc. have timer controlled spray systems.
Depending upon size of facility or need, one centralized dispensing/distribution system can be installed or separate systems can be installed throughout the facility.
Emergency shut-off control will be installed in each treatment room and a master in the facilities control room.
Rapid drying solutions will be used throughout the system.

Public Bars and Restaurant Locations:
System utilizes electronic motion detectors and occupancy detection systems to activate system in common areas such as hallways and lobbies.
System is mounted above ceiling and only spray heads penetrate the ceiling tile, much like a sprinkler system.
Primary activation system is a push button control located in the room that is pushed when all other cleaning of room has been accomplished. The push button is protected by a lockable cover plate with select personnel having the key.
System can also be controlled remotely via a computer based system that can be programmed to activate a single room or several rooms or a complete system spray.
System can be programmed to activate based on motion sensors and occupancy detectors so room can be sanitized just before opening of the facilities and after all other cleaning has been done.
Controlled spray releases can be manually provided to key areas of the bar and food prep areas, such as sinks, countertops, cooler doors, etc.

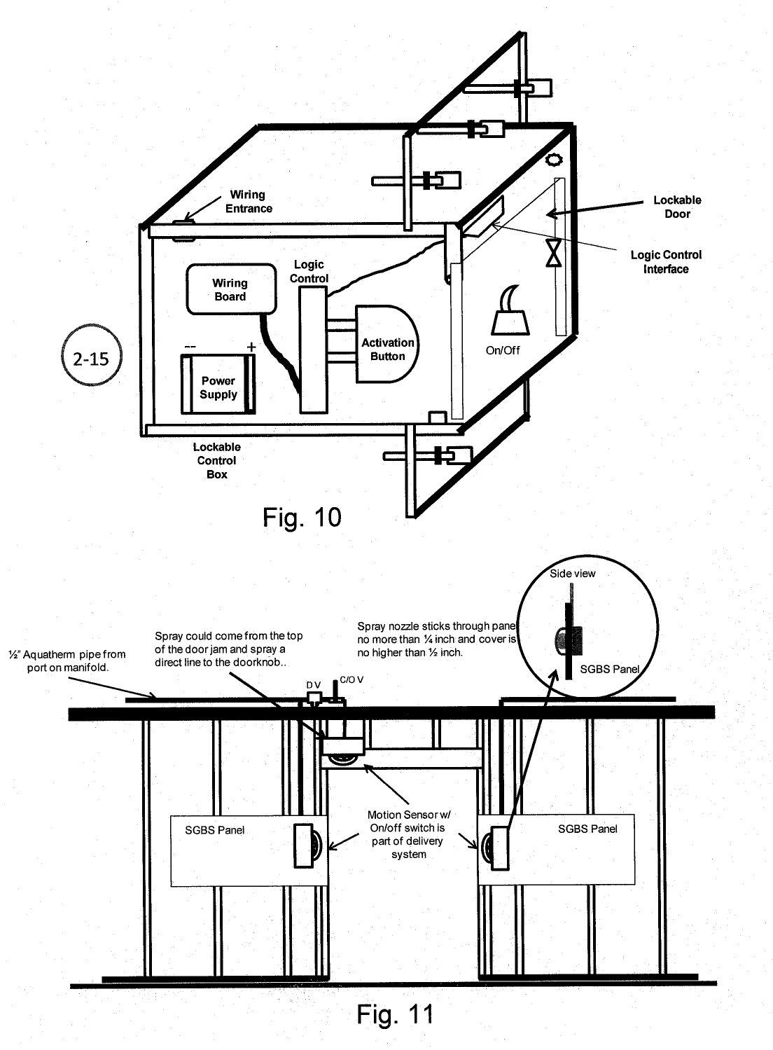

Elevator and Escalator Systems (New and Retrofit) & Revolving Doors (FIG. 10):
Elevator and revolving door system's uses electronic motion detection and room occupancy detection systems to control the output of the spray nozzle.
The systems are designed to be an automatic sanitizing system, but can be manually controlled.
System sanitization is allowed only when the elevator or revolving door is unoccupied.
SGBS Escalator system sanitizes not only the handrail but the steps as well.
The handrail system is mounted on both sides of the escalator and sprays the handrail as it makes the underside trip.
A fan blower is utilized to help in the drying process prior to handrail returning to the customer access area.
The steps also proceed through a spay system under the escalator and are dried prior to returning to the customer area.
Sanitization system is completely hidden from the customer's view.
System can be controlled manually, remotely or via a computer controlled activation system located at the escalator or in a facilities office.
A protective cover over the distribution system prevents overspray.
The solution used in the system is a fast drying sanitization solution.

Hotel/Motel Lobbies and Common Areas:
System utilizes electronic motion detectors and occupancy detection systems to activate system in common areas such as hallways and lobbies.
System is mounted above ceiling and only spray heads penetrate the ceiling tile, much like a sprinkler system.
Depending upon size of hotel/motel, the dispensing system can be centralized or separate systems per floor.
System can't be overridden while occupancy detectors sense people in the area.
System can be controlled via a centralized computer system located in the facilities office, or each dispensing/distribution system can be manually operated.
Only a fast drying sanitizing solution will be used.
With a room button activation system installed with the system, each room can be sanitized once the maid service is completed, or as the room occupant wishes.

Shopping Carts:
Enclosed spraying area through which shopping carts are pushed.
Shopping cart is sprayed with sanitizing solution as it passes through the enclosed area.
Spray is triggered by a pressure activated switch located in the wheel channel. As chat is pushed/pulled into the spray area, the cart's wheels will roll over the pressure switch, thus activating the spray control system.
A separate area within the enclosed system dries the carts, using high pressure fans, before they return to the customer area.
A motorized conveyor is used to push/pull the carts through the spraying/drying area of the enclosure.
The system is self-contained and can be installed in existing cart-return area at the entrance of the store. This system can be de-installed and moved to another area with ease.

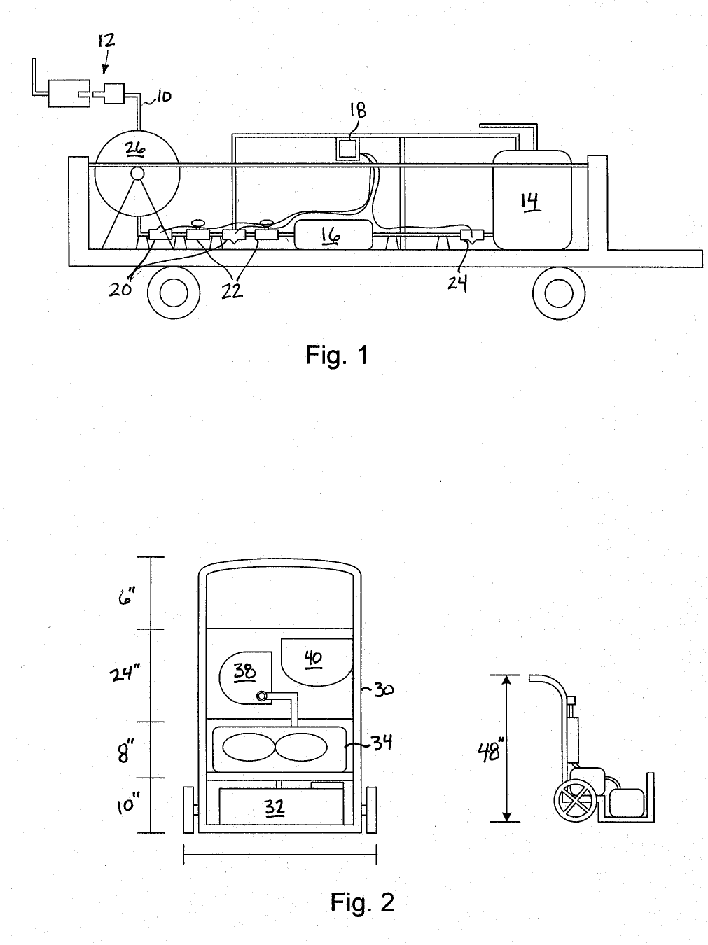

Mobile Germinator System (see FIGS. 1, 2)

General Description of Each Unit:

Basic Unit-Transportation Industry Version was developed to provide a rapid method of disinfecting various passenger carrying transportation systems, such as; Buses, Airplanes, Ships, Trains, Taxis, Limo's, Vans, Trams, Subways, etc. This system will be especially useful in disinfecting school buses before children have been picked up and after they have been dropped off at school or home. Mass transportation systems, such as city buses and taxis are another primary target to help eliminate germs, viruses, etc.

The basic unit is pulled behind a vehicle (gas or electric) up to the vehicle being disinfected, or in some cases the vehicle being disinfected can be brought to the Basic Unit. All vehicles being disinfected will have been converted with the spray dispensing system as shown in the appropriate diagrams below.

The operator will reel off what ever amount of hose 10 is needed to reach the input port on the vehicle being disinfected and connect the “quick connect/disconnect” system 12 to the input port of the vehicle being disinfected. Once hose has been connected to vehicle being disinfected, the operator shall start the Mobile Germinator System and bring pressure up to required level. Once pressure level is steady at right level, the operator will engage the control unit to begin the disinfecting process. The basic unit will provide a measured amount of disinfecting agent over a specified timeframe controlled by an electronic timer. The correct amount of agent will be based upon USDOT, USEPA, State and Local agencies, as well as foreign government environmental agencies. Once disinfecting is complete, the operator will disengage the hose, re-wind it and move on to the next vehicle to be serviced.

The Basic Unit-Transportation Industry Version consists of a 50 to 200 gallon liquid holding tank 14, a bladder tank to help develop pressure and allow for drain-off, either an electric or gas operated motor driven liquid pump 16 capable of handling bleach or other disinfecting products, control unit 18, pressure gauges 20, pressure relief valves 22, drain valves 24, 300 feet of clear poly-braid 1 hose mounted on an electric re-wind hose wheel 26, with quick connect/disconnect system mounted on outer end, 5 KW auxiliary generator, 20 gallon auxiliary gas tank, standard metallic sealed, lockable tool box(s), mounted on a standard 6½ foot×8, single or axle open trailer with tailgate.

The 50/50 Unit-Sanitizer and Mildew/Mold Eradicator System is a trailer or truck mounted system, while the 200/200 Unit-System is a trailer only based system, that was developed to provide normal day-to-day or emergency response to many facets of the private and public need to remove mildew, mold, and disinfect areas that come in contact with humans on a regular basis. These systems provide the capability to not only provide everyday cleaning and disinfecting capabilities, but also provide emergency response to major disasters such as Hurricanes, Tornados, Floods, Fire, etc.

During new construction or remodeling where the unit is taken down to the bare studs, the Sanitizer and Mildew/Mold Eradicator can be used to spray the entire stud system from top to bottom to remove any mildew and mold build up that has occurred. Then just before the sheetrock or other wall covering is installed, the entire area would be disinfected.

After a major disaster, such as a Hurricane, both the 50/50 and the 200/200 Unit would be deployed to assist home owners, small businesses, hospitals, restaurants, or any other location that was flooded for any period of time. The 200/200 unit is just a larger version of the 50/50, and meant to handle larger jobs such as larger apartment complexes, small malls, hotels, motels, etc.

When the unit, either the 50/50 or the 200/200, arrives at the requested job site, the operator will reel off the amount of hose necessary to access all areas within the damaged facility. The interior wall coverings would have been removed and the area where flood waters stood would be exposed and have dried out. The operator shall spray the mildew/mold eradicator over the entire area, ensuring all exposed areas are thoroughly soaked with the eradicator. If the flood waters had been standing in the area for more than a few days, it is recommended that the area be treated twice over a two day period. The same is recommended for areas that had water damage caused by rain, sprinklers, etc. Once treatment is completed, the operator shall thoroughly soak the entire interior with the disinfectant.

The only technical difference between the 50/50 (trailer or truck mounted) unit and the 200/200 unit is the size of the liquid container used. They consist of the appropriate sized liquid holding tank, gas operated motor driven liquid pump capable of handling bleach products, control unit, pressure gauges, pressure relief valves, drain valves, 300 feet of clear poly-braid 1 inch hose mounted on an electric re-wind hose wheel, with a hand-held sprayer attached to the end of the hose and group of different sized nozzles. There is a 5 Kw auxiliary generator, 10 gallon fuel tank for the 50/50 units and a 20 gallon fuel tank for the 200/200 unit. The 50/50 unit will be mounted on a standard 6½′×8′ single axle trailer or the platform can be mounted in the back of a standard pick-up truck bed, while the 200/200 unit will be mounted on a dual axle, 6½ feet×12′ utility trailer.

A smaller unit may be mounted on a hand truck or cart (FIG. 2). In one embodiment, the cart 30 has a 2-gallon tank 32 for storing a disinfecting solution. The disinfecting solution is pumped by an electric pump 34 through a hose that is stored on a hose holder 38. A spray nozzle at the end of the hose can be stored on a corresponding holder 40

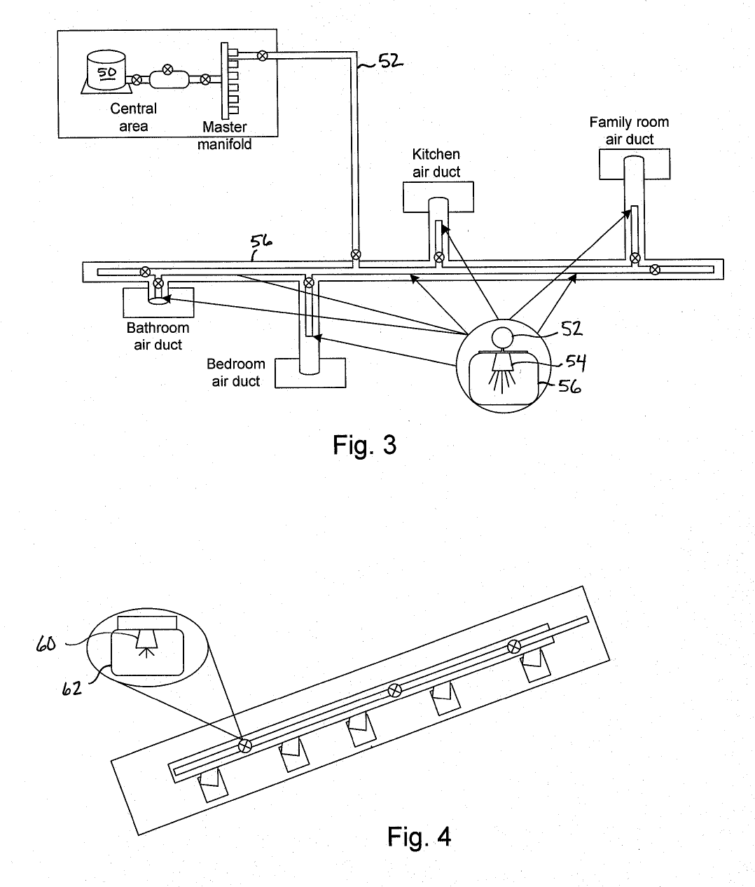

Air Duct Spray System (See FIGS. 3, 4)

The components of the Home Air Duct Spray System (FIG. 3) are the same as for any other part of the Germinator System used in a house or small office; Aqua-Therm Pipe, Male “T” Connectors, Pipe Caps, 90-degree elbows, Pipe Straps, Washers, & Lock Nuts, controllable cut-off valves and dumb drain valves, controllable solenoids, regular pipe “T”, etc. are used to pump disinfecting solution from a storage tank 50, through piping 52 to various spray nozzles 54 positioned at different locations with the air ducts 56 of the HVAC system. Similarly, in a system installed in an aircraft or other mass transit vehicle (FIG. 4), disinfecting solution can be pumped to spray nozzles 60 mounted in the ducts 62 of the air distribution system.

Select an open port on the Master Manifold. Measure the full route of the Air Duct Spray System and assemble an equal amount of Aqua-Therm Pipe. Determine the number of 90-degree turns in the system and gather them. Determine the number of Air Vents to be serviced, as well as the number of other insertion points along the air duct path. Gather the required number of Male “T” Connectors. For each Male “T” Connector, you will need two flat washers, 1 lock washer and one small nylon lock nut.

Beginning at the Master Manifold, prepare the pipe and connect a solenoid, then a controllable drain and then a controllable cut-off valve. Secure these in position. Now begin adding the pipe along the route selected. (Following the general concept shown in FIG. 3), secure the pipe to the air duct system. At each point selected to insert a spray nozzle, drill the appropriate sized hole. As pipe is install to that area, fuse the Male “T” connector to the pipe, connect a mounting plate to the male portion of the connector and secure it with the washers and lock nuts. Now insert the nozzle through the hole and secure it using small self-tapping screws or aluminum tape. At key points as indicated on FIG. 3, install a controllable cut-off valve and a dumb drain valve. Continue this process until all spray nozzles have been installed.

Connect each monitor/control point within the route to an individual logic control unit point within the controller, using the appropriate sized wiring according to the manufacturers' recommendations. Label both ends of the wiring, by labeling the logic end with the originating point and at the originating point to which the logic point is connected.

Once all pipe and wiring has been completed, perform an overall system visual check to make sure everything is in accordance with the installation instructions. The system is now complete and ready for testing prior to final activation.

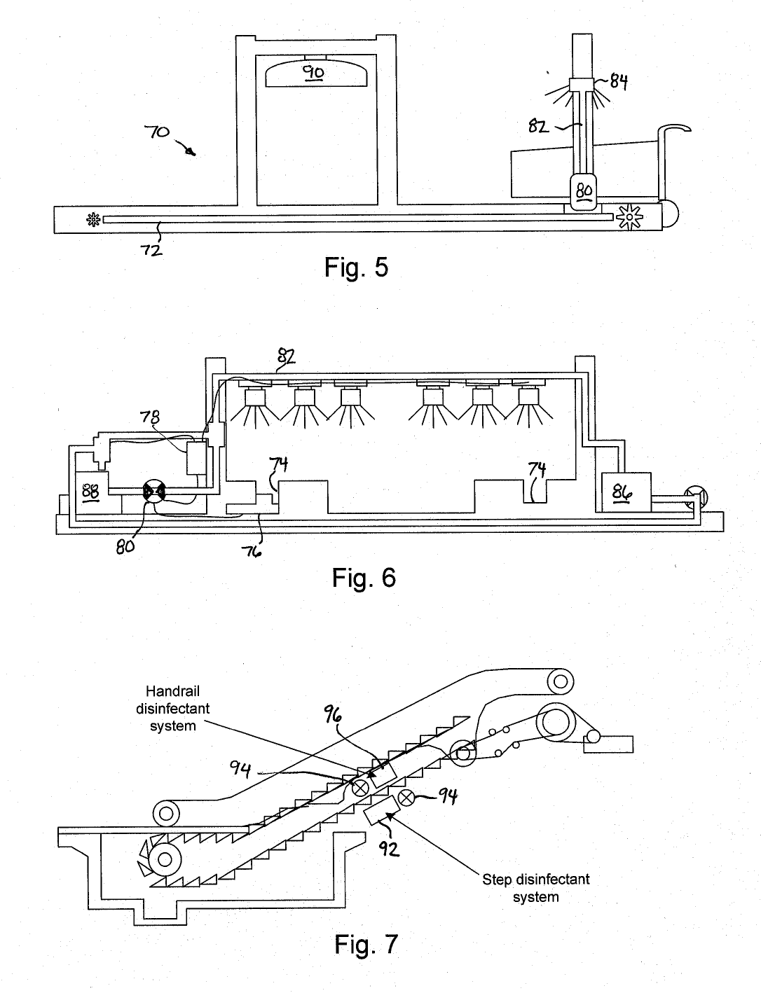

Shopping Cart Sanitizing System (See FIGS. 5, 6)

Shopping carts are found in almost every shopping mall, department store, food market, auto parts stores, etc. and they all have one thing in common; they are constantly being handled by humans, and we all know that means Germs being transferred from one person to the next through the hand contact with the shopping cart's handle.

Several supermarket-type stores have a tube of sanitizing towels located next to the shopping cart area for their customers to use to wipe down the handle area of the shopping cart. This requires the customer to pull out one of the wet wipes, wipe down the cart's handle area, and then dispose of the wet wipe. Usually there is no place to dispose of the wipe so they end up on the floor or in another shopping cart for some else to remove.

When the customer pulls the wet wipe from its container, they end up with the sanitizing solution all over their hands and maybe their clothes, and no dry towels to wipe hands off with. Although most customers would like to clean the cart, they also don't want to walk around the store with this sanitizing solution on their hands, so they just by-pass the cleaning and go about their merry way.

What if there was a better way, a way that would sanitize the entire cart, not just the handle, and the customer did not have to handle the sanitizing solution at all. Not used wet wipes to pick up from the floor or out of the carts, no paper towels for Drying the customer's hands and not wet hands for the customers. SGBS has the answer—An Automated Shopping Cart Sanitizing System that can be installed anyplace that shopping carts are currently in use or are to be used.

The system 70 is a chain 72 or belt driven conveyor system designed to have the wheels of the shopping carts fit into two slots 74 on the rack. The conveyor will catch the wheels with blocks and begin to drag it along the rack. The wheels of the carts will activate either an electronic eye or a pressure switch 76 that sends a signal to the control box 78, which sends a signal to the pump 80.

The pump will activate and send a sanitizing solution along the piping system 82. The control box will also send a signal to the spray nozzles 84 to open and the pressure from the pump will spray the solution onto the shopping cart as it rides below. Any overflow solution is captured in a secondary holding tank 86 and then pumped back to the primary output tank 88. After a set time, the pump will shut-off and the spray nozzles will close. As the cart continues along the rack a large blower fan 90 will assist in drying the carts before they are returned to the customer waiting area.

Shopping cart wheels are slotted in the guides and the left wheels will depress the activation button, that will send a signal to the Control Box, which will send a signal to the pump to turn on for a set time limit, thus sending the sanitation solution through the lines to the spray heads. The control box will activate the spray heads to spray the solution onto the Shopping Cart while it is pulled along the rack. Any excess solution will be captured in the over-flow tank and since it is still clean solution, shall be pumped back to the output tank.

After a set time, the pump will shut off and the spray heads will close. The shopping carts will continue along the rack, where a large blower will assist in the drying of the carts. The carts will continue along the rack until they reach the end and are returned to the cart waiting area.

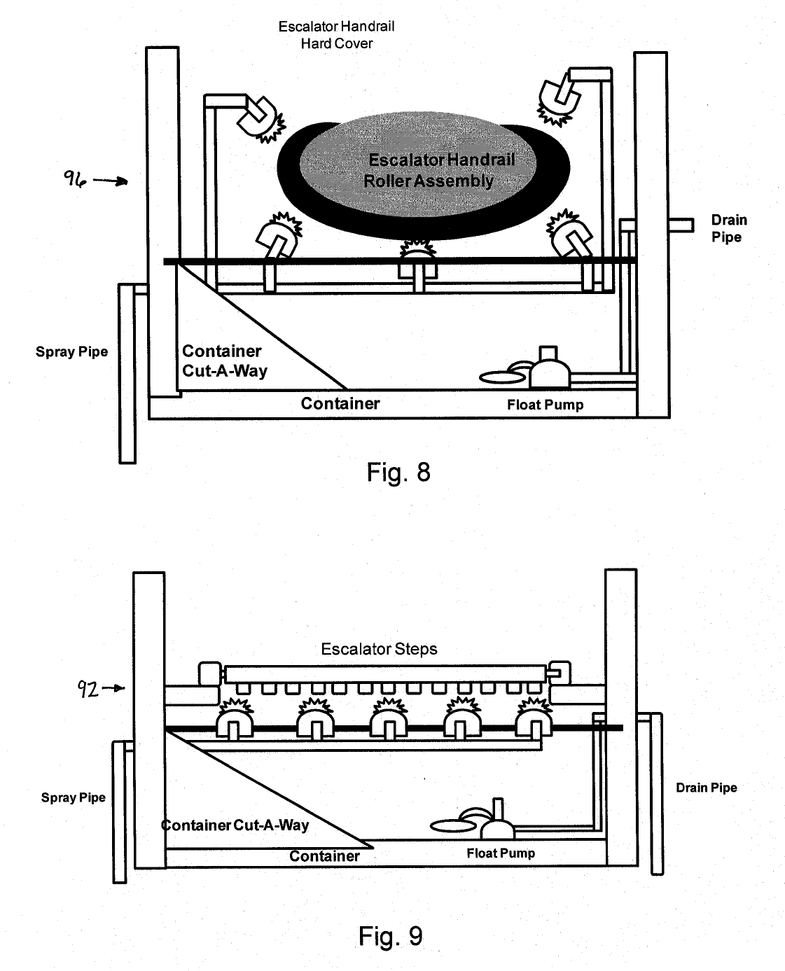

Escalator Sanitizing System (FIGS. 7-9)

The use of a liquid, quick drying disinfecting agent dispensing system, built-in the design of new Escalators seems to be a better and more economical method for new construction. The idea would be to have the handrails and steps pass through a continuously spraying assembly that sprays a fine mist over the entire handrail hard cover and the top sides of the steps with the disinfecting agent.

To insure the ability of operating an escalator either as an Up or Down unit, the disinfecting system (96, 92) would be installed near the center of the escalator. The handrail units would be mounted in the center area. Since escalators can be run either as an up or a down unit, a pickup flange will have to be fitted to both sides of the disinfectant container so as to lift the steps to a flat position as they go through the disinfecting area.

As the handrail and steps begin their return trip, the multiple sprayers would apply the disinfectant to the surfaces. As they continue the return trip under the escalator, the disinfectant would have time to dry off. As a final assurance, a continuously running, low watt air blower 94 would help dry the disinfectant.

A float pump located in the disinfectant container will sense when the excess mist falling back into container has began to reach a pre-set level and thus begin to pump it out to a safe holding tank until it can be removed and safely gotten rid of in accordance with acceptable EPA, local, state and federal laws. Since this liquid is considered contaminated, it cannot be reused. The holding tank, which would be located under the Escalator in a convenient, out of the way spot, is equipped with a drain valve and cut-off valve. A separate holding tank would contain the disinfectant in the same area, under the escalator.

Test2

Public Restrooms

Any where you have a tremendous amount of people interacting on a daily basis, especially world travels, you are subject to come in contact with any number of germs, viruses, various colds and flu's, and things you can't even recognize. So you see people walk up to a door and instead of just reaching out and opening it, they'll take out a napkin or handkerchief to put between their hand and the doorknob (Does anyone know where that handkerchief was last. You'll see it a lot coming out of bathrooms where people have a paper towel and open the door and then throw paper towel on the floor, behind the door.) No . . . Staying at home or wearing a haz-mat suit is not the answer, at least in most people's minds.

These areas can never be 100% germ free environments, but they can be a whole lot more healthy for everyone, if on a regular basis, they are subjected to a spraying of disinfectant and germicide agents. This spraying should come as a general air disinfecting, as well as a direct assault on the most offensive areas, such as sinks, urinals and water closets.

Basically with most current locations and all new locations, the ability to add a air disinfecting system is rather simple. It is the attacking of the most offensive areas that creates the challenge. This can be accomplished through some sort of retrofit when dealing with existing locations to adding changes to the design for new facilities.

Until manufacturers can accommodate the health thinking audience with restroom furnishings that have built-in sprayers and other disinfecting systems, add-ons will have to be the answer.

The upgrading of design plans during initial construction is the simplest and most cost effective method and does not require a lot of discussion, as it is rather simple and straight forward. The Aqua-Them tubing is ran through the interior walls of the new construction with breakouts for the spray heads. These units would be adjustable so as to be able to be directed directly at the area needing sanitizing. As with a retrofit system, it will be computer logic controlled and can be controlled either automatically or manually. The spray nozzles would be able to cover the entire sink area, faucets to tops & bowls, the complete urinal unit as well as the complete water closet, especially where these items have to be touched.

The heart of the system will be the Germinator Disinfecting System. For now we will concentrate on the Retrofit concept and to cover the entire Restroom facility, top to bottom and every thing in between, even those tiny little cracks that germs and viruses can hide in and you can't even see. An overhead pipe disinfecting and sanitizing system would be deployed in a large public restroom. A downsized version of this would handle lesser sized facilities. Depending whether the ceiling was an completely open version or had a false ceiling, would determine the installation approach. In an completely open version, pipe hangers could be suspended from the actual ceiling to hold the piping in a grid layout, much like the fire sprinkler system. In a false type ceiling, the same approach would be used to hang the piping, however, ceiling tiles would have to be cut in numerous locations to allow the spray-head to protrude through the tile. The system would be an automatic timer release with a manual override. Spray-heads would be controlled through hard wire connections to logic control units wired back to the Master Controller in the Control Room. The system would automatically release a fine mist into the facility on a times basis, such as 5 seconds, every 10 minutes. During heavier periods of use, the spray interval could be varied to better accommodate the needs of the facility. An Air Duct Sanitizing System could be employed in the Restroom areas as well. This system induces the disinfecting/sanitizing agent in the air flow through spray heads installed inside the air duct system. All of this is controlled by a Master Controller. Retrofit devices would be installed for direct coverage of water closets and urinals, as well as the faucets, counter tops and sinks. These are the most used and most in need of disinfecting and sanitizing on a very regular basis. These units would have a directed spray to the most contaminated areas of the restroom. They are totally controlled through the IR occupancy detectors and commands from the Master Controller as to when to release the disinfecting and sanitizing agent. In some cases, it may take more than one spray head device to cover the required area, in which case, a second or even a third would be used.

SGBS Home & Office Controller

This is a touch screen, computer based System. The home & office controller can be sized to fit just about any size home or small office.

Monitor & Control Points: 20 to 260

Emergency One Touch Buttons: 4 to 8

Favorite Buttons: 4 to 20

Camera: 1 to 5

The number of monitor/control points can be added by incorporating the next panel of 20 points. The emergency buttons allow for one touch calling to the fire, police, ambulance service, hospital, national poison hotline, drug overdose hot line, etc.

Favorite buttons can be programmed to allow one touch calling of favorite places like: doctor's office, dental office, pizza and sandwich shops, dry cleaners, movie theaters, restaurants, home delivery shopping, etc.

The system is designed to allow both monitoring of the multiple access points as well as providing timed and instant control over key elements of the system. For example, the system will allow for timed released of disinfecting solution after someone enters/exits a main door, or that point can be overridden to allow for immediate disinfecting of the doorknob.

As smart appliances and electronics are added to the house, they will be incorporated into the overall home controller system. In some cases, these new elements may be added to two different sections, depending upon actions required. Points within the system can be moved between touch screen locations via the keyboard entry. All actions of the home & office controller can be remotely accessed via your mobile device or from your office computer via the internet or wireless connections. Security of the system is password controlled and has built-in firewall protection for the internet access. The system can be located anywhere the homeowner or office manager selects. For security or ascetic reasons, it could be mounted behind a wall covering or picture that would swing open to reveal the Controller System.

In the event of a technical problem within the Controller System, a manual override switch allows the system to be shutdown until it can be repaired or replaced. Since the data that has been entered into the system from start-up is stored in the computer based portion of the system, and the Controller panel is the human interface point, the Controller panel can be disconnected and replaced quickly, so as to allow rapid repair of the system. The Controller panel can then be taken back to the shop for repair and then returned to the owner.

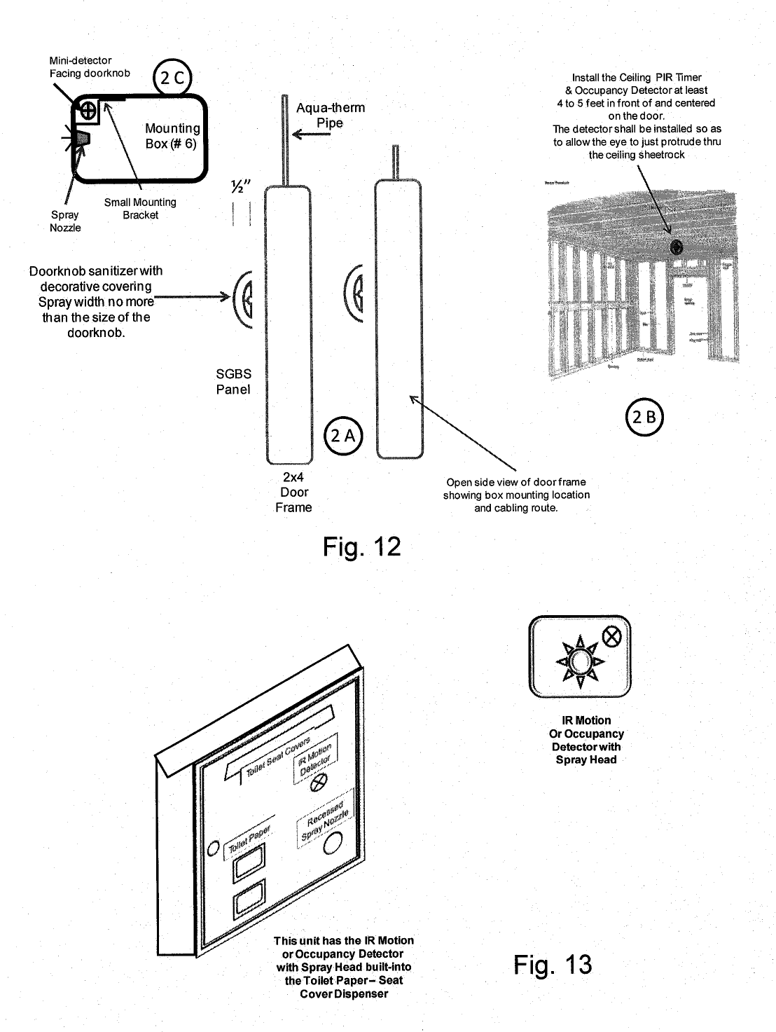

An Example of a Description of a Doorknob Sanitizer (FIGS. 11-12):

An apparatus for sanitizing a doorknob to reduce or eliminate germs that are left on the doorknob when a person touches it to open or close the door. In one embodiment, the apparatus includes a container for sanitizing liquid, a spray nozzle for spraying the sanitizing liquid on the doorknob, and a conduit for carrying the sanitizing liquid from the container to the spray nozzle. The apparatus also includes a sensor for detecting that a person has touched the doorknob, as well as a pump mechanism that pumps sanitizing liquid from the container to the spray nozzle, where the sanitizing liquid is sprayed onto the doorknob. The pump mechanism is triggered in response to the sensor detecting that the doorknob has been touched. In one embodiment, the sensor may sense that the doorknob has been turned or otherwise moved with respect to the door. In another embodiment, the sensor may be an inertial sensor that detects lateral movement of the doorknob (i.e., opening or closing of the door) without movement of the doorknob with respect to the door. The pump mechanism may be designed to delay its activation for some predetermined interval of time after the sensor detects that the doorknob has been touched in order to avoid spraying the sanitizing liquid on the person.

In one embodiment, the container for the sanitizing liquid is positioned apart from the door, and a tube connects the container to the pump mechanism. The container may be a visible unit that is positioned against a wall, or it may be integrated into in a wall or a ceiling, or it may be placed in another convenient location. In another embodiment, the container may be integrated into the door itself in order to eliminate the need for tubing between the moving door and a stationary container. Tubing may nevertheless be used to connect the door-based container to the pump and spray nozzle. The door-based container may be externally mounted to the door, or integrated into the door so that it is not visible. In one embodiment, the spray nozzle is integrated into the doorknob itself. The nozzle may direct the spray of sanitizing liquid upward so that a mist falls back down onto the doorknob, or it may be positioned externally (e.g., on the door above or to the side of the doorknob) so that the mist of sanitizing liquid is sprayed onto all or part of the doorknob.

### UV Embodiment 1

### Small & Portable Device Sanitizing System

Sanitizing Wands allow you to slowly wave the wand over whatever item you want to sanitize. This requires you to stand around, holding the unit in one hand and slowly moving it over the item. Usually, the height of the wand varies, since it is hard to keep the wand at one single height. There is a better way that uses a Small and Portable Device Sanitizing System. This is a unit that is just slightly larger than your standard Microwave Oven and weighs about the same as well. The unit will allow you to sanitize items such as your Phone, iPad, Tablet, Laptop, books, kitchen utensils or anything else that will fit into the unit. For ease of operation, the unit loads from the top and not the front. It runs from a standard 120V, 20 A circuit. This unit uses the same UV technology that the wands use, but with more powerful light intensity, and automated exposure in an enclosed compartment, so there is no more standing around and moving the wand manually.

Many people are aware of the normal effects of UV light—Sunburn, which is very harmful to the skin. But there are different levels of UV light and it has been found that some ranges of UV light kills bacteria, viruses, fungus, and other germs.

Ultraviolet (UV) light ranging from 200-280 nm is classified as UV-C. This range of the UV spectrum has a germicidal effect on bacteria and viruses. UV does not affect moisture or temperature of food and is economical. UV treatments have the advantage in that no excessive protection for individuals is necessary and that no residual radioactivity occurs, even at high levels of exposure. The exposure of bacteria, viruses and spores to UV rays alter the bonds with the DNA double helix, that results in either mutation or lethality to cells. UV light is effective in air, liquid or on surface treatments. UV is commonly used to disinfect surfaces on packaging or in food processing environments.

Now, UV light is being used to sanitize many more things. It is used by many hospitals, doctor's and dentists. Even most barbers have stopped using the old solutions to sanitize their combs and scissors. They now just pop them under UV light for a period of time and out come sanitized tools of the trade.

The present system is very user friendly. Simply open the top of the sanitizing chamber, place in the chamber whatever device you wish to sanitize, close the top and lock it. Now, using the control panel, set the time you want the unit to run by selecting an on time and an off time. Push the start button and walk away until the system signals that it has completed the requested cycle, by ringing a buzzer or providing similar audible or visual alerts.

Once activated, sanitizing UV-C lamps on the interior of the system will come on and remain on until the selected amount of time has expired. (NOTE: Even though the lights are off, they may still be hot, so use caution removing your item(s).

The UV Light Chamber is non-transparent to UV-C light, thus containing the light within the unit. UV-C can be harmful if you stare directly into the light or keep it on your skin for long periods of time. NEVER look directly into the light.

A safety system senses whether the chamber is closed. The safety system will automatically shut off the UV-C light sources if the chamber is opened while the UV lights are activated.

The UV Light Chamber will measure a minimum of 24 inches wide, 24 inches deep, and 24 inches high.

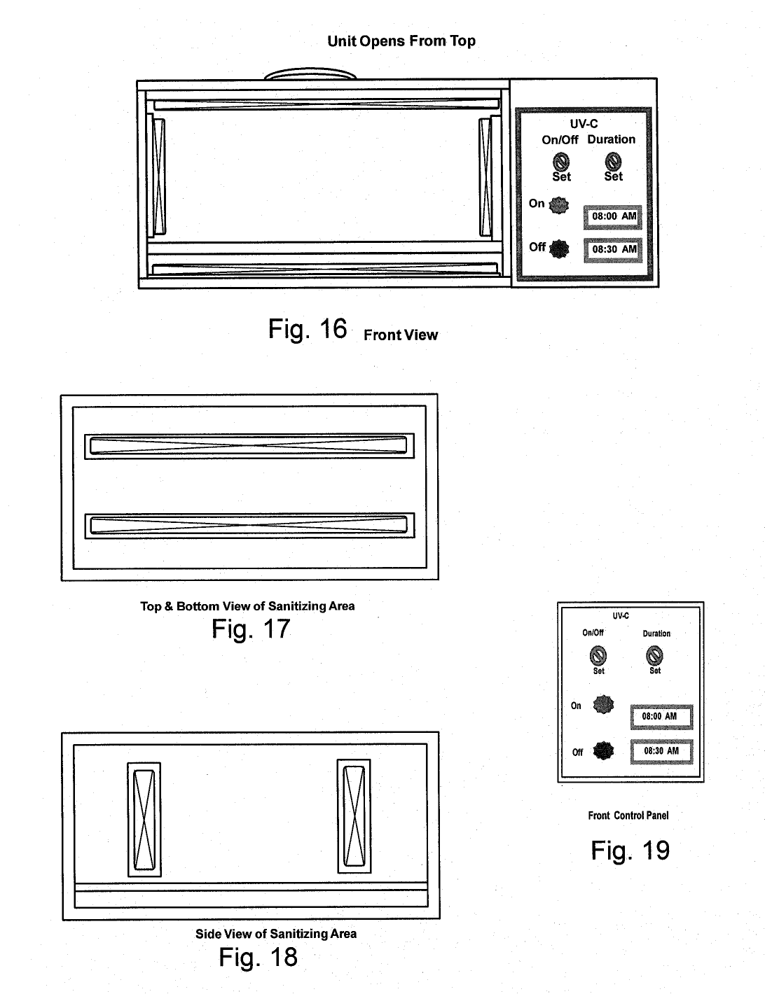

FIGS. 16-19 illustrate this embodiment of the sanitizing system. FIG. 16 depicts a front view of the sanitizing system, showing the front control panel and the general positioning of the UV-C light sources within the sanitizing chamber. FIG. 17 shows the positioning of the UV-C light sources at the top and bottom of the sanitizing chamber. FIG. 18 shows the positioning of the UV-C light sources at the sides of the sanitizing chamber. FIG. 19 shows an exemplary control panel that can be used to set the exposure time for items placed in the sanitizing chamber.

The countertop food sanitization system has an appearance similar to that of a typical microwave oven, however it uses UV light to perform the sanitizing of food stuffs. The UV-C range of the UV spectrum has a germicidal effect on bacteria and viruses. UV does not affect moisture or temperature of food and is economical. UV treatments have the advantage in that no excessive protection for individuals is necessary and that no residual radioactivity occurs, even at high levels of exposure. The exposure of bacteria, viruses and spores to UV rays alter the bonds with the DNA double helix that results in either mutation or lethality to cells. UV light is effective in air, liquid or on surface treatments. UV is commonly used to disinfect surfaces on packaging or in food processing environments.

Food stuffs are placed in a clear bowl provided with the unit and placed inside the sanitizing unit for the specified timeframe. Once the sanitizing is completed, take the food stuffs out and wash them prior to placing them in your refrigerator, or other storage unit. The unit has two control panels; (1) a quick reference guide for certain fruits and vegetables, (2) A control panel that can be set by the operator. The times can be adjusted from one (1) minute to fifteen (15) minutes, depending upon what is being sanitized. A general reference chart is provided with each unit. The unit may have buttons that are pre-programmed to operate that unit for specific times corresponding to selected foods.

The system is activated from the front control panel and allows the user to set the number times the system is turned on and off, how long the system will remain on when active, what times during the day it will be on. The user can set the system to come on between one (1) and five (5) times each 24 hours period and remain on from 1 minute to 15 minutes each time.

Once the bowl has been placed in unit and the door closed, the control panel can be activated. Once activated, the sanitizing UV-C lamps will fill the inside of the unit with the bacteria/fungus/spore killing light. To ensure complete coverage of the food stuffs inside the unit, the bowl is placed on a turntable that will rotate the entire sanitizing cycle.

Once it has completed, the UV lights will shut off (caution—they may still be hot) and the door can be opened and food stuffs taken out. Wash the items immediately and store properly. The food stuffs can be sanitized again prior to actual use.

FIGS. 16 and 19 indicate the control panels located on the front of the unit.

Cleaning of the unit is simple. Close the door, activate lights for five (5) minutes. Once bulbs have cooled down, wipe inside with hot, damp cloth.

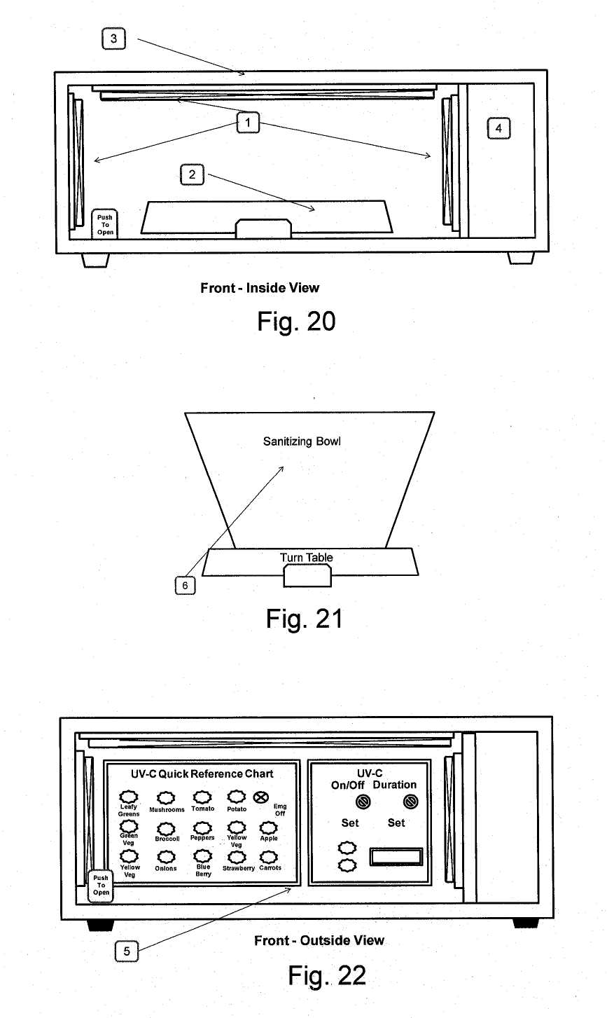

In one embodiment, the system includes the following components, which are depicted in FIGS. 20-22:

| | |
|---|---|
| 1. Turn-Table System | 1 each |
| 2. UV-C Light System | 6 each |
| 3. Sanitizing System Housing | 1 each |
| 4. Electronic & Motor Unit | 1 each |
| 5. Control Panels | 2 each |
| 6. Holding Bowl - Large | 1 each |

The unit may be constructed as follows. This unit is to be manufactured by a licensed and UL approved manufacturer having experience with the manufacturing and construction of microwave ovens.

The system consist of an outer housing unit, made of non-UV-transparent materials, so as to eliminate the UV-C light from escaping the sanitizing chamber, a motor and electronic section, turntable, UV-C light chamber and a large, clear bowl for placing the food stuffs in for sanitizing, and the control panels.

Safety systems built into unit will not allow the UV lights to come on when door to unit is open. The system must be serviced by a trained and licensed Technician.

### Embodiment 2

### Countertop Sanitizer System

The Countertop Sanitization System has the appearance of an under counter mounted lighting system. However, this automated system, when activated, will swing into action and sanitize counter tops using UV-C light, thus killing bacteria. Viruses, fungus and mold spores unseen by the human eye.

UV light is effective in the treatment of surface such as countertops and vanities. UV is commonly used to disinfect surfaces on packaging or in food processing environments, and is safe for use throughout the home as a sanitizing system.

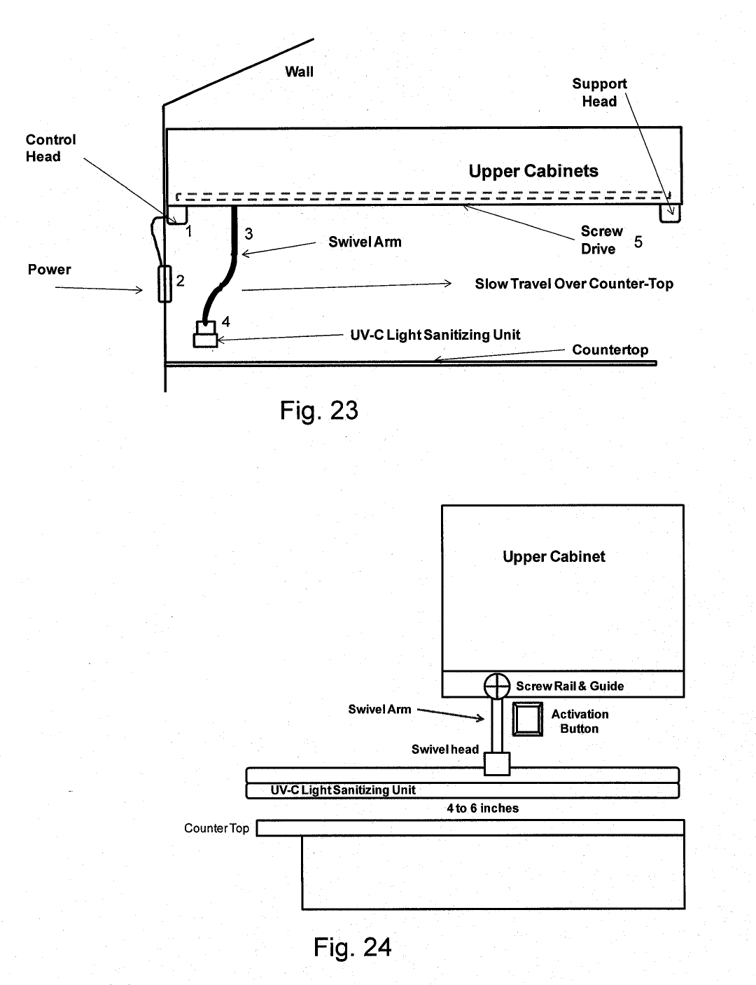

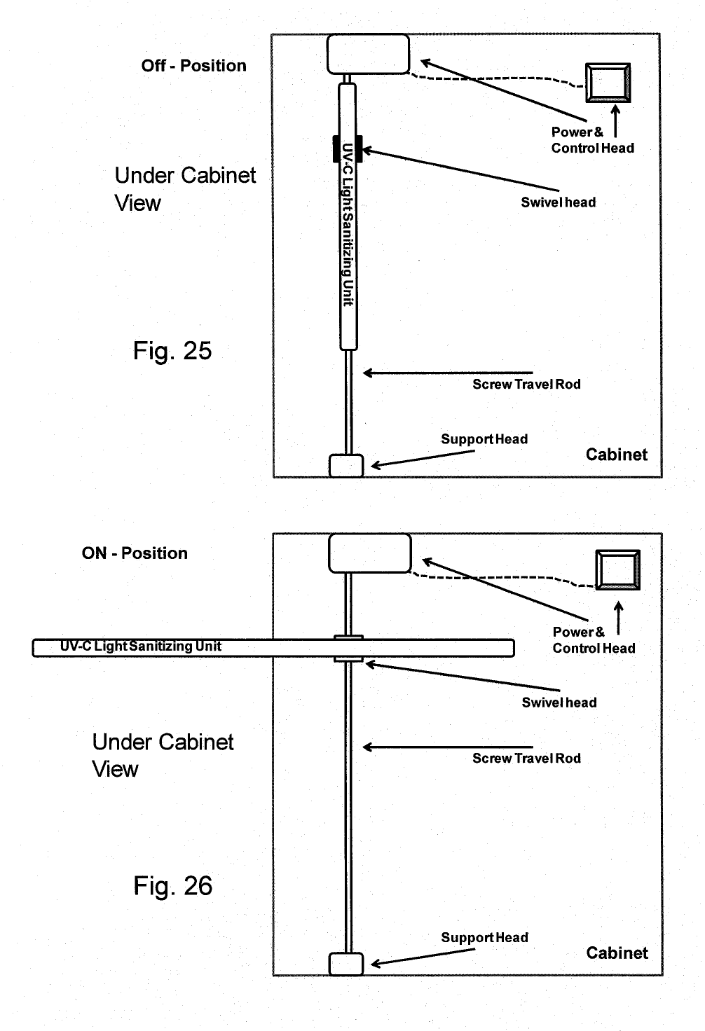

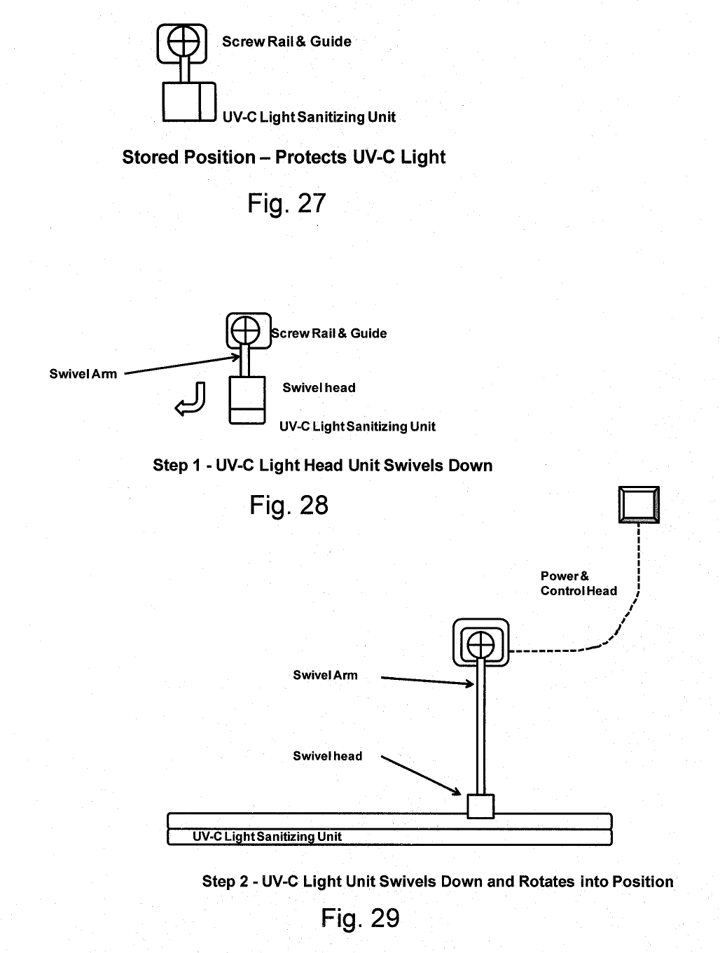

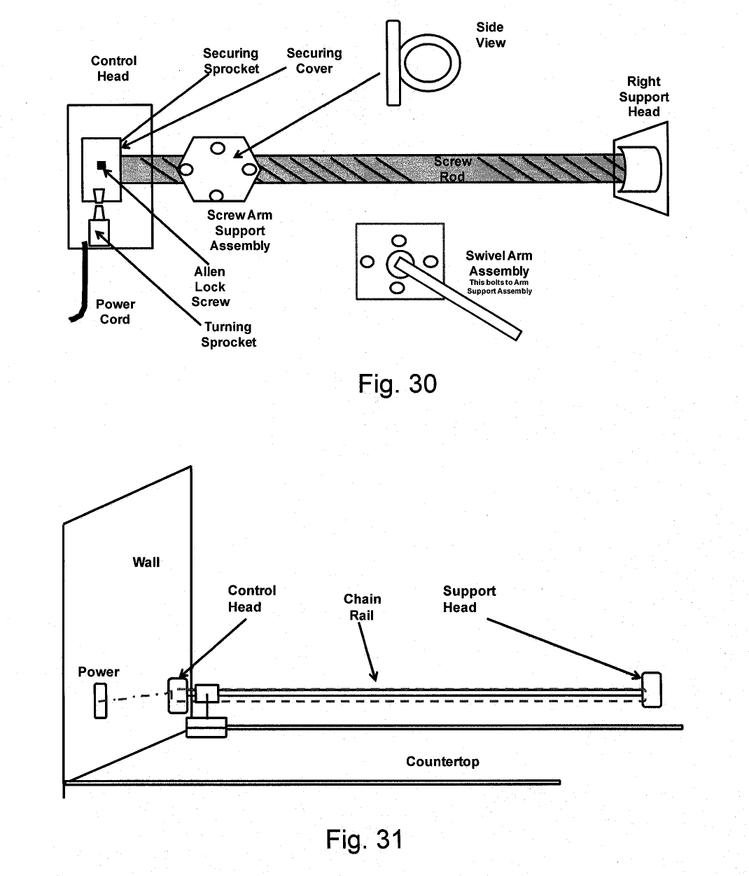

The countertop sanitization system is designed to be installed under the upper cabinets, or along the wall. FIGS. 23-31 show the under cabinet design and how the system actually works. For standalone counter islands, the unit could be built into the back of the island and activation would open a covered area so that the arm will swing out in the same manner as in the standard system.

The system is activated by pushing the activation button located on the wall. This will power the unit on, and the UV-C Light Sanitizing System will rotate from its stored and safe position to its operating position above the countertop.

Once activated, the swivel arm positions the UV-C light head unit within 2 inches of the wall and between 4 and 6 inches above the countertop. Once the unit is in position, the UV-C light will activate and the unit will begin a slow travel above the countertop, until it reaches the end of the track. When it reaches the end of the initial sweep, the unit will begin a return sweep to the far end.

Once it has completed the forward and return sweeps, then unit will go into the shut down mode, when the light is turned off, the UV-C Light unit rotates back to storage and safe position and the unit then retracts to under the cabinet and out of the way.

The travel time for the unit can be varied as per the homeowners desires. Times will range from 2 minute to 10 minute sweep. FIG. 31 indicates the mounting of the unit along the wall.

In one embodiment, the system includes the following components, which are depicted in FIG. 23:

| | |
|---|---|
| 1. Control Head | 1 each |
| 2. Power Head | 1 each |
| 3. Swivel Arm to Unit | 1 each |
| 4. UV-C Light Sanitizing Unit | 1 each |
| 5. Screw Drive to Arm | 1 each |

The unit may be installed as follows. Mark the mounting location under the cabinet for the screw drive unit and the support heads. Now install the right support head at the far end of the cabinet. Now, in accordance with the instruction, measure the length from the support head to the opposite end. Now mark the screw rod using this length minus 1 inch and cut. Screw the arm support assembly onto the screw rod at least one foot from left end. Now insert the right end of rod into the right support assembly and using the small level, make sure screw arm is level and mark center location at left end on wall. Using the center mark, align the left control head center guide and secure to wall. Now attach the securing nut to the left end and align with holding bracket inside of the Control head and slide into place. Place cover over nut and secure.

Now bolt the Swivel Arm Assembly to the Screw Arm Assembly, making sure the entire arm is extended and in the normal scan position. Inside the control head are adjustment set/lock screws that provide for final adjustments of the system. One of these will be a speed adjustment for how fast you want the scan to move across the countertop.

Now plug unit into power and turn unit on. Following the instruction in the assembly package, adjust each screw until the unit is in the stored position. This will complete the alignment and the unit is now ready for testing. Turn the power off to the unit and wait 10 seconds. Now turn power back on and the green ON LED should be lit. If, not double check power and troubleshoot. Once the ON LED is lit, the unit is ready to scan the countertop.

Push the scan button on the control head and the unit will swing down into the scanning position and after five (5) seconds, the unit will begin to scan the countertop at the speed set in the final adjustments.

The scan is a forward and back scan. If you set time for 5 minute scan, the forward scan will be two and a half minutes and the back scan will be two and a half minutes. At end of scan unit returns to store position.

Once you are satisfied with the complete operation of the unit, place the control head cover on the unit and it is now ready for repeated use.

### Embodiment 3

### Refrigerator Crisper Sanitizing System

UV light can be used to keep fruits, vegetables and meats fresher, longer, without degrading these foods or presenting any harmful side effects. UV-C light has been proven to extend the life of fruits and vegetables from the field to the home. Killing these harmful bacteria and other germs/viruses will also extend the life of meats.

The Refrigerator Food sanitization system is installed in the vegetable crisper and meat storage units of the refrigerator. Each of these units has its own sanitization system and is separately controlled by the front door control panel.

The system is activated from the front control panel and allows the user to set the number of times the system is turned on and off, how long the system will remain on when active, what times during the day it will be on. The user can set the system to come on, for example, between one (1) and five (5) times each 24 hours period and remain on from 1 minute to 15 minutes each time. Each compartment can be set on its own independent schedule.

Once activated, the sanitizing UV-C lamps will illuminate the food items with sanitizing light for the period of time that was chosen by the homeowner. When the cycle is over, the lights will automatically turn off until the next cycle and then repeat as many times as chosen.

The time for the unit can be varied as per the homeowner's desires, but should be guided by the industry recommended sanitizing times per the provided chart.

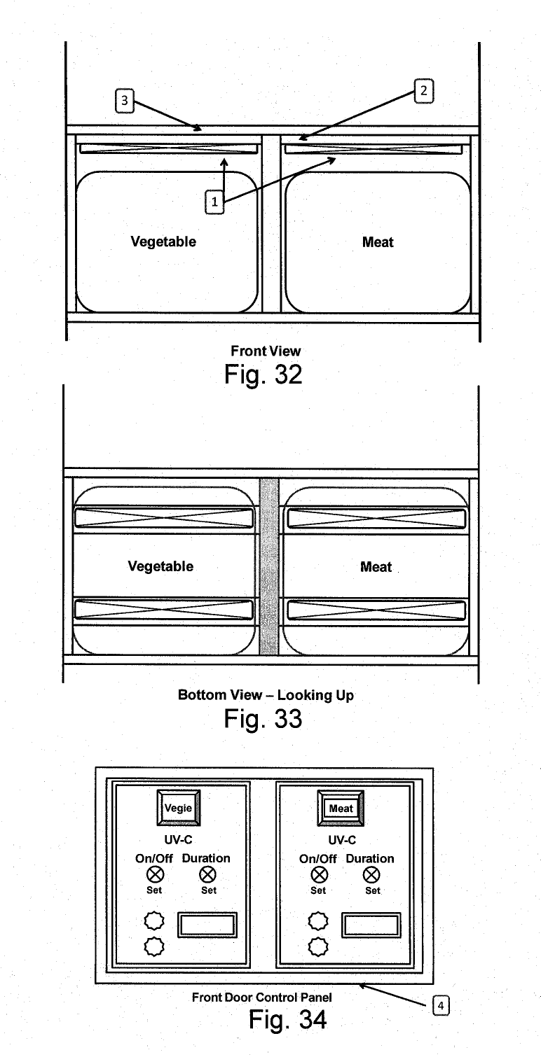

In one embodiment, the system includes the following components, which are depicted in FIGS. 32-34:

| | |
|---|---|
| 1. UV-C Light Bulbs | 4 each |
| 2. UV Light Bulb holders | 4 each |
| 3. UV-C Light Container - 2 Drawer | 1 each |
| 4. UV Control Panel - 2 Section | 1 each |

The refrigerator crisper sanitizing system is factory installed and tested. The manufacturer may make a retrofit unit at a later date for existing units.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the claims. As used herein, the terms “comprises,” “comprising,” or any other variations thereof, are intended to be interpreted as non-exclusively including the elements or limitations which follow those terms. Accordingly, a system, method, or other embodiment that comprises a set of elements is not limited to only those elements, and may include other elements not expressly listed or inherent to that embodiment.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention as detailed herein.

What is claimed is:

1. A sanitizing system comprising:
a tank that contains a sanitizing liquid;
a pipe system that is coupled to the tank, thereby enabling fluid from the tank to flow into the pipe system;
a pump coupled to the tank and the pipe system, wherein the pump pumps the sanitizing liquid from the tank to the pipe system; and
a plurality of spray nozzles coupled to the pipe system, wherein at least a portion of the sanitizing liquid that is pumped into the pipe system is forced through the spray nozzles, wherein sanitizing liquid that is dispensed through the spray nozzles is applied to one or more disinfecting areas.

2. The sanitizing system of claim 1, wherein the tank, the pump and at least a first portion of the pipe system are mounted on a transportable mobile unit.

3. The sanitizing system of claim 2, wherein the transportable mobile unit comprises a trailer, and wherein the first portion of the pipe system includes a quick-connect/disconnect fitting that enables the first portion of the pipe system to be alternately connected to and disconnected from one or more second portions of the pipe system that include the spray nozzles and are permanently installed in the disinfecting areas.

4. The sanitizing system of claim 3, wherein the one or more second portions of the pipe system are installed in one or more vehicles, wherein the disinfecting areas comprise interior cabins of the vehicles, wherein the first portion of the pipe system is alternately connectable to different ones of the vehicles to sanitize the interiors of the vehicles.

5. The sanitizing system of claim 4, wherein the one or more second portions of the pipe system are installed in air distribution ducts in the one or more vehicles.

6. The sanitizing system of claim 1, wherein the tank, the pump, and the pipe system including the nozzles are mounted on a mobile unit that is transportable to the disinfecting areas, wherein the disinfecting areas comprise individual rooms in one or more buildings.

7. The sanitizing system of claim 1, further comprising a frame having one or more tracks that accommodate wheels of shopping carts, wherein the tracks constrain the shopping carts to travel through the one or more disinfecting areas.

8. The sanitizing system of claim 7, further comprising: one or more sensors that detect the shopping carts; and a control system that is coupled to the one or more sensors and controls at least one of the pump, the pipe system and the spray nozzles based on information received from the one or more sensors, thereby dispensing the sanitizing liquid into the one or more disinfecting areas when occupied by the shopping carts.

9. The sanitizing system of claim 1, wherein the spray nozzles are mounted in proximity to an escalator, wherein the spray nozzles dispense the sanitizing liquid onto at least one of a handrail of the escalator and a set of steps of the escalator, wherein the at least one of the handrail and the set of steps continually pass through the one or more disinfecting areas while the escalator is in operation, thereby continually disinfecting the at least one of the handrail and the set of steps.

10. The sanitizing system of claim 9, further comprising one or more blowers, wherein the blowers are positioned in proximity to the escalator, wherein the blowers are directed to a portion of the escalator so that the at least one of the handrail and the set of steps are dried by the blowers after the sanitizing liquid is dispensed onto the at least one of the handrail and the set of steps.

11. The sanitizing system of claim 1, wherein the spray nozzles are mounted in proximity to a door, and wherein the spray nozzles dispense the sanitizing liquid onto a door handle of the door, the system further comprising one or more sensors that detect use of the door handle, the system dispensing the sanitizing liquid onto the door handle in response to detecting that the door handle has been used.

12. A sanitizing system comprising:
an ultraviolet (UV) lighting element;
a container in which the UV lighting element is enclosed, wherein the container has a chamber therein in which an article to be disinfected can be positioned and wherein the container is non-transparent to UV light; and
a control panel coupled to the UV lighting element, wherein the control panel controls the UV lighting element to illuminate an article in the chamber according to one or more predetermined settings.

13. The sanitizing system of claim 12, wherein the UV lighting element is disabled when the container is open.

14. The sanitizing system of claim 12, wherein the container comprises an enclosure within a refrigerator.

15. A sanitizing system comprising:
an ultraviolet (UV) lighting element;
a movable arm on which the UV lighting element is mounted; and
a sweeping mechanism coupled to the movable arm, wherein when the sweeping mechanism is activated, the UV lighting element is activated and the sweeping mechanism sweeps the movable arm above a countertop with the UV lighting element in close proximity to the countertop, thereby illuminating the countertop with UV light and disinfecting the countertop.

16. The sanitizing system of claim 15, wherein the sanitizing system is mounted to the underside of a cabinet that is above the countertop, wherein the sweeping mechanism extends downward from the cabinet to a position above the countertop.

17. The sanitizing system of claim 16, wherein when the sweeping mechanism is not activated, the movable arm is retracted away from the countertop to a position that is in close proximity to the underside of the cabinet.
